(19) United States
(12) Patent Application Publication
(10) Pub. No.: US 2013/0023842 A1
(43) Pub. Date: Jan. 24, 2013

(54) MEDICAL VACUUM SEALING DRAINAGE DEVICE

(71) Applicant: Wuhan VSD Medical Science & Technology Co., Ltd., Wuhan (CN)

(72) Inventor: Jiuhong SONG, Wuhan (CN)

(73) Assignee: WUHAN VSD MEDICAL SCIENCE & TECHNOLOGY CO., LTD., Wuhan (CN)

(21) Appl. No.: 13/625,024

(22) Filed: Sep. 24, 2012

Related U.S. Application Data
(63) Continuation-in-part of application No. PCT/CN2011/072066, filed on Mar. 23, 2011.

(30) Foreign Application Priority Data
Mar. 23, 2010 (CN) 201010132585.4
Jun. 29, 2010 (CN) 201010221569.2
Jun. 29, 2010 (CN) 201020252540.6
Jan. 10, 2011 (CN) 201120005386.7

(51) Int. Cl. A61M 1/00

(52) U.S. Cl. 604/319

(57) ABSTRACT

A medical vacuum sealing drainage device, including: a porous foam soft pad (1); a drainage tube (2) including a tail end; and an elastic sealing rubber film (3). The drainage tube (2) is arranged in the porous foam soft pad (1) and the tail end thereof extends outside the porous foam soft pad (1). The elastic sealing rubber film (3) is arranged on an outer side of the porous foam soft pad (1). A sealing opening (3a) for allowing the drainage tube (2) to extend out is formed on the elastic sealing rubber film (3). A diameter of the opening portion of the sealing opening (3a) is less than the outer diameter of the corresponding extending pipe.

### CROSS-REFERENCE TO RELATED APPLICATIONS

This application is a continuation-in-part of International Patent Application No. PCT/CN2011/072066 with an international filing date of Mar. 23, 2011, designating the United States, now pending, and further claims priority benefits to Chinese Patent Application No. 201010132585.4 filed Mar. 23, 2010, Chinese Patent Application No. 201020252540.6 filed Jun. 29, 2010, Chinese Patent Application No. 201010221569.2 filed Jun. 29, 2010, and to Chinese Patent Application No. 201120005386.7 filed Jan. 10, 2011, the contents of which, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 14781 Memorial Drive, Suite 1319, Houston, Tex. 77079.

### FIELD OF THE INVENTION

The invention relates to a medical vacuum sealing drainage device, which is suitable for drainage of wounds on the body surface and for vacuum sealing drainage in a body cavity.

### BACKGROUND OF THE INVENTION

A vacuum sealing drainage technology (VSD for short) was invented by Dr. Wim Fleischman at the University of Ulm Germany in 1992 and theory thereof has been formed, and it is for drainage of wound surface of limbs. In 1994, Chinese professor Qiu Huade first applied the VSD technology to general surgical departments, and initiated application of the VSD technology in the general surgical departments.

A basic configuration of the conventional VSD technology includes a vacuum source (including a medical suction unit, a central vacuum unit, or a vacuum drainage bottle used in a hospital), a drainage pipe, polyvinyl alcohol foams orother medical multi-hole foams, sponges or gauzes (a multi-hole foam cushion for short), a breathable film for adhesion and sealing (a sealing film for short), and a drainage container. The vacuum source is connected for drainage, whereby conducting liquefaction materials of body tissues such as exudation, cataclysm, liquefaction necrosis tissue fragments, pus and so on into the drainage container.

In use, the drainage pipe with multiple side holes is wrapped with the multi-hole foam cushion, and disposed on the wound surface or in a wound cavity. The sealing film is used to tightly seal the multi-hole foam cushion and an outlet of the drainage pipe whereby separating them from the outside. Then the drainage container is connected, and finally the vacuum source is connected, and thus a drainage system is formed. Since vesicles in the multi-hole foam cushion are connected to each other and are rich in flexibility, negative pressure can reach every point of a targeted drainage area whereby forming omnibearing drainage. Under the action of the negative pressure, comparatively big, tender and blocky educts are cut and molded into granules, which enter the drainage pipe via openings of the multi-hole foam cushion or vesicles connected to each other, and then are quickly inhaled into the drainage container. Big educts that may block the drainage pipe are stopped by the multi-hole foam cushion, adhered to the surface of the multi-hole foam cushion, and left a body of the device along with the multi-hole foam cushion as drain is removed or replaced. Closing of the sealing film is a critical factor to maintain negative pressure operating as drainage force, and to separate the drainage area from the outside, which effectively prevent pollution and cross infection.

However, during use of the conventional VSD technology in drainage of a surface wound tissue, several problems exist: 1) sealing thereof is inconvenient. Good sealing is a key for ensuring drainage effect and the most difficult step during the whole vacuum sealing drainage. Common-used sealing methods include: a method of stabbing holes, a mesentery method, a suture method, a dumpling method, and so on. However, the above-mentioned methods are very complex during practical operation, and operative doctors can grasp them after repeated operations and practices. In addition, infirm sealing often causes air leakage, and further decreasing of treatment effect or even treatment failure of the VSD system. 2) VSD materials would be dehydrated and become dried and hard, if being under a negative pressure for a long time. 3) Sealing cannot be achieved, when the system is used for the drainage of orthopedics or perineum.

### SUMMARY OF THE INVENTION

In view of the above-described problems, it is one objective of the invention to provide a medical vacuum sealing drainage device. The medical vacuum sealing drainage device is good in sealing effect and simple in structure, convenient to use, and not only suitable for drainage of wounds on the body surface, but also suitable for vacuum sealing drainage in a body cavity.

To achieve the above objective, in accordance with one embodiment of the invention, there is provided a medical vacuum sealing drainage device, comprising: a porous foam soft pad, and a drainage tube arranged in the porous foam soft pad and provided with a tail end extending outside the porous foam soft pad. An elastic sealing rubber film is arranged on an outer side of the porous foam soft pad; a sealing opening for allowing the drainage tube to extend out is formed on the elastic sealing rubber film; a diameter of an opening part of the sealing opening is less than an outer diameter of a corresponding extending pipe.

In a class of this embodiment, the sealing opening used for allowing the drainage tube to extend out and formed on the elastic sealing rubber film is a circle hole, the diameter of which is 0.3-4 mm less than the outer diameter of the corresponding extending pipe.

In a class of this embodiment, the sealing opening used for allowing the drainage tube to extend out and formed on the elastic sealing rubber film is a pipe orifice, and the diameter of the opening part of the pipe orifice is 0.3-2 mm less than the outer diameter of the corresponding extending pipe.

In a class of this embodiment, when the sealing opening used for allowing tubes to extend out and formed on the elastic sealing rubber film is a pipe orifice, the pipe orifice takes a shape of an inverted horn, the diameter of the opening part of the pipe orifice is 1-2 mm less than the outer diameter of the drainage tube, and the diameter of the bottom of the pipe orifice is 2-6 mm more than the outer diameter of the drainage tube.

In a class of this embodiment, when the sealing opening used for allowing tubes to extend out and formed on the elastic sealing rubber film is a pipe orifice, an included angle between the sealing opening and a main body of the elastic sealing rubber film is between 15 and 90°.

In a class of this embodiment, a plurality of small holes is formed on the side wall of the drainage tube.

In a class of this embodiment, a single or a plurality of drainage tubes are employed; the plurality of drainage tubes are arranged evenly with a distance of 25-35 mm between each other, and connected through a multi-way connector in the tail end.

In a class of this embodiment, a flushing pipe is arranged in the drainage tube, one end of the flushing pipe extends to the inside of the drainage tube, the other end of the flushing pipe is led out from the side wall at the tail end of the drainage tube, and a sealing cover is arranged at the tail end of the flushing pipe.

In another embodiment of the invention, there provided is an orthopedic vacuum sealing drainage device, comprising: a porous foam soft pad, and a drainage tube arranged in the porous foam soft pad and provided with a tail end extending outside the porous foam soft pad. A first elastic sealing rubber film is arranged on the outer side of the porous foam soft pad, a sealing opening for allowing the drainage tube to extend out is formed on the first elastic sealing rubber film, the diameter of the opening part of the sealing opening is smaller than the outer diameter of the corresponding drainage tube; a second elastic sealing rubber film and a third elastic sealing rubber film are arranged at puncture needles connected with an orthopedics external fixation device, vertical pipe orifice shaped sealing openings are formed on the second and third elastic sealing rubber films, and sleeved on the two puncture needles respectively; the second and the third elastic sealing rubber films are placed on the first elastic sealing rubber film or directly placed on the porous foam soft pad for sealing in a covering manner.

In a class of this embodiment, when the sealing opening used for allowing the drainage tube to extend out and formed on the first elastic sealing rubber films is a pipe orifice, an included angle between the pipe orifice and a main body of the elastic sealing rubber film is between 15 and 90°.

In a class of this embodiment, the vertical pipe orifice shaped sealing opening on the second and the third elastic sealing rubber films takes a shape of an inverted horn, the diameter of the opening part of the pipe orifice is 0.3-2 mm less than the outer diameter of the puncture needles, and the diameter of the bottom of the pipe orifice is 2-6 mm more than the outer diameter of the corresponding puncture needles.

In a class of this embodiment, a flushing pipe is arranged in the drainage tube, one end of the flushing pipe extends to the inside of the drainage tube, the other end of the flushing pipe is led out from the side wall at the tail end of the drainage tube, and a sealing cover is arranged at the tail end of the sealing cover.

In another aspect, the invention provides a vacuum sealing drainage device used at the perineum, comprising a porous foam soft pad, and a drainage tube arranged in the porous foam soft pad and provided with a tail end extending outside the porous foam soft pad. A first elastic sealing rubber film is arranged on the outer side of the porous foam soft pad, a sealing opening for allowing the drainage tube to extend out is formed on the first elastic sealing rubber film, and the diameter of the opening part of the sealing opening is smaller than the outer diameter of the corresponding drainage tube; the vacuum sealing drainage device used at the perineum further comprises an anus inserting catheter, a second elastic sealing rubber film is arranged at the position where the anus inserting catheter penetrates a vacuum area of the vacuum sealing drainage device, and provided with a vertical pipe orifice shaped sealing opening, the vertical pipe orifice shaped sealing opening is sleeved on the anus inserting catheter, and the second elastic sealing rubber film is placed on the first elastic sealing rubber film or directly placed on the porous foam soft pad for sealing in a covering manner.

In a class of this embodiment, an annular rubber air-filled or liquid-filled bag is arranged on the outer side at the front end of the anus inserting catheter; an air or liquid guide pipe parallel to the anus inserting catheter is also arranged; the front end of the air or liquid guide pipe is communicated with the rubber air-filled or liquid-filled bag, the rear end of the air or liquid guide pipe extends outside the rear end of the anus inserting catheter, and a sealing cover is arranged at the rear end of the air or liquid guide pipe.

In a class of this embodiment, a flushing pipe is arranged in the drainage tube, one end of the flushing pipe extends to the inside of the drainage tube, the other end of the flushing pipe is led out from the side wall at the tail end of the drainage tube, and a sealing cover is arranged at the tail end of the flushing pipe.

Advantages of the invention are summarized below:
(1) The problem of air leakage of the extending part of the drainage tube is solved thoroughly, the stable and successful vacuum sealing drainage is ensured, the key technology for vacuum drainage is overcome, the using effect is better, and the possibility of air leakage of the VSD (vacuum sealing drainage) system is greatly reduced, that is, the conventional air leakage rate is reduced to 5% from 60%.
(2) Due to the arrangement of the elastic sealing rubber film, the device is suitable for drainage of wounds both on the body surface and in the body cavity. Two sets of different conventional drainage devices (drainage of wounds both on the body surface and in the body cavity) are combined into a set.
(3) The operation is more convenient, for the wounds both in the body cavity and on the body surface, the extending part of the drainage tube is sealed through the elastic sealing rubber film, thus the sealing of the sealing film is more simple and easier; the defects of complex structure and heavy load of doctors due to the adoption of the VSD technology for the wounds in the body cavity are overcome. During the drainage process of wounds in the body cavity, an effective support (carrier) is provided for the sealing film, so that the embarrassment that in the radical mastectomy, Miles operation for rectal cancer, radical operation for vaginal tumor, osteomyelitis fenestration, lavage and drainage operation and perineum explosion injury operation, the drainage tube is not provided with a support during the film sticking process, so an opposite sticking method has to be adopted is avoided. The sealing film is only required to be sealed around the elastic sealing rubber film, thus the drainage operation for the wounds in the body cavity is simple, and the sealing effect is good. The complexity to operators during the use of the film sticking method and the harm of the hole poking method to the normal body tissue of patients are avoided; the oppression of the normal tissues around during the drainage at the side outlet hole is avoided.
(4) During the drainage of wounds on the body surface, besides providing a sticking film supporting surface, the elastic sealing rubber film also can covers the porous foam soft pad by increasing its area, so that the problem that the if sealing film is directly stuck on the porous foam soft pad, the porous foam soft pad may become hard due to the evaporation of water in the porous foam soft pad through the sealing film, and then the vacuum sealing drainage will fail is avoided.
(5) The drainage tubes are connected with the catheter through the multi-way connector, the absorption area is increased through a plurality of rows of small holes on the side wall of the drainage tube, the outlet of each drainage tube can be used as both a drainage opening and a flushing opening for flushing a blocked pipe, and both the drainage and flushing are convenient.
(6) The dedicated flushing pipe is used for flushing the blocked pipe, thus the retrograde infection is avoided; the dedicated flushing pipe also can be used for diluting, soaking and softening the drainage in the porous foam soft pad in due time, thus the drainage is more convenient; the operation time is reduced, the pain of patients is alleviated, and the wound healing time of patients is shortened.
(7) The orthopaedic vacuum sealing drainage device changes the operating instructions and rules of the traditional orthopaedic external fixation technology and combines the sealing and fixing operation, the orthopaedic external fixation device is hermetically sealed, so that the problem of failure or reduced effect in vacuum sealing drainage treatment due to air leakage is eliminated, the infection at the puncture needle is also eliminated, the key technology of applying the vacuum sealing drainage device to orthopaedic trauma is overcome, the infection at the orthopaedic trauma puncture needle is basically eliminated, the frequent condition that infection extends to the bone cavity along the puncture needle is completely avoided, the risk of treatment of patients is greatly reduced, the pain of patients is eased, the treatment effect is effectively ensured, and the disability rate of patients is greatly reduced.
(8) According to the vacuum sealing drainage device used at the perineum, because the inserting catheter for the perineum is additionally arranged, in the VSD (vacuum sealing drainage) treatment, the normal physiological excretion of patients is not affected, the excrement is effectively prevented from overflowing to pollute the wound, the incidence rate of infection is effectively lowered, and the wound healing time of patients is reduced.

### BRIEF DESCRIPTION OF THE DRAWINGS

The invention is described hereinbelow with reference to accompanying drawings, in which:

FIG. 1 is an overall structure diagram of a first basic vacuum sealing drainage device;

FIG. 2 is an overall structure diagram of a second basic vacuum sealing drainage device;

FIG. 3 is a diagram of a medical vacuum sealing drainage device used inside the body;

FIG. 4 is a diagram of a sealing opening of an elastic sealing rubber film taking a shape of a circular hole;

FIG. 5 is a diagram of a sealing opening of an elastic sealing rubber film taking a shape of a pipe orifice;

FIG. 6 is a structure diagram taken from line A-A of FIG. 5;

FIG. 7 is a diagram of a vertical pipe orifice shaped sealing opening on an elastic sealing rubber film;

FIG. 8 is a diagram of a drainage tube and a two-way connector mechanism for two drainage tubes;

FIG. 9 is diagram of four drainage tubes and a four-way connector mechanism which are arranged in a porous foam soft pad respectively;

FIG. 10 is an overall structure diagram of an orthopedic vacuum sealing drainage device;

FIG. 11 is a longitudinal sectional view of FIG. 10;

FIG. 12 is an overall structure diagram of a vacuum sealing drainage device used at the perineum; and

FIG. 13 is a structure diagram of an anus inserting catheter.

### DETAILED DESCRIPTION OF THE EMBODIMENTS

For further illustrating the invention, experiments detailing a vacuum sealing drainage device are described below. It should be noted that the following examples are intended to describe and not to limit the invention.

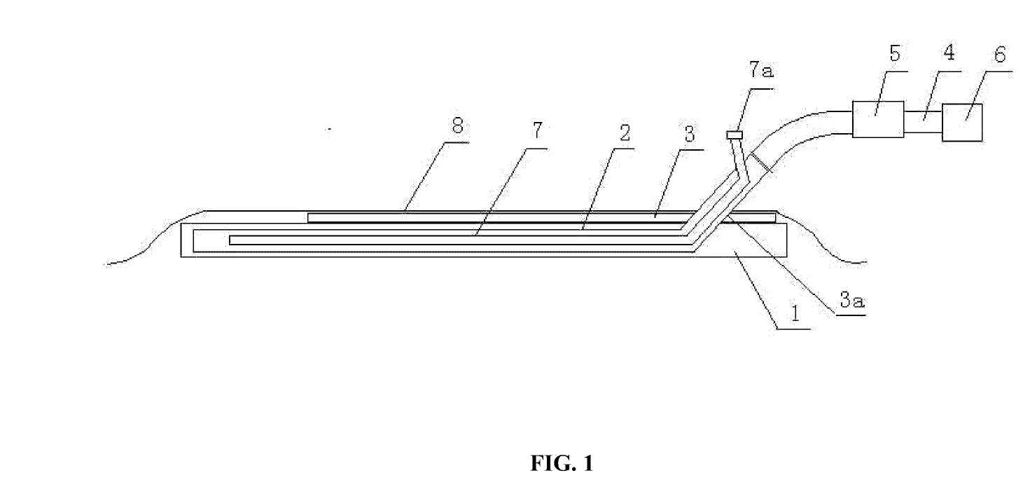

FIG. 1 is an overall structure diagram of a first basic vacuum sealing drainage device, which comprises:

a porous foam soft pad 1, a drainage tube 2 arranged in the porous foam soft pad 1 and provided with a tail end extending outside the porous foam soft pad 1, and an elastic sealing rubber film 3 for allowing tubes to extend out is arranged on the outer side of the porous foam soft pad 1. A sealing opening 3a for allowing the drainage tube 2 to extend out is a circular hole and formed on the elastic sealing rubber film 3.

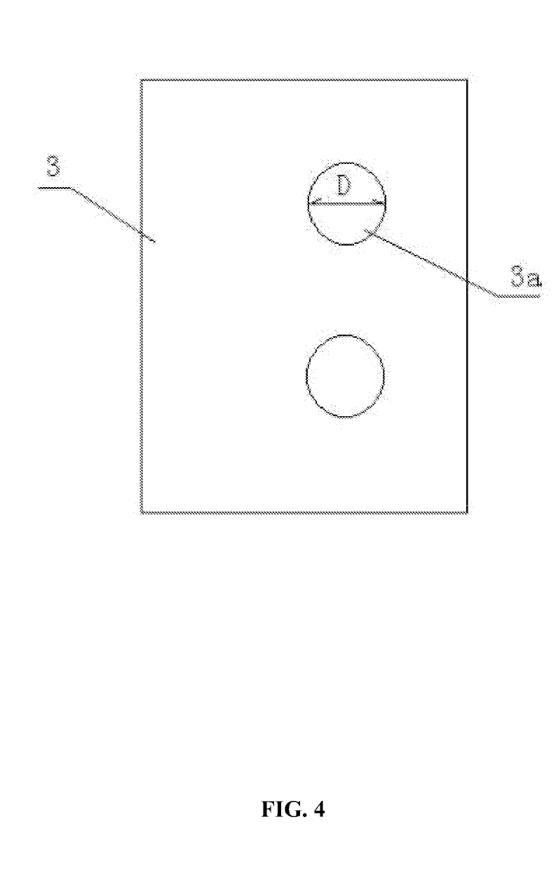

FIG. 4 is a diagram of the elastic sealing rubber film. The diameter D of the sealing opening 3a is 0.3-4 mm less than the outer diameter of the drainage tube 2.

Due to the elasticity of the elastic sealing rubber film, the sealing opening 3a is tightened on the drainage tube 2 for sealing. During the using process, a sealing film 8 is resealed on the elastic sealing rubber film 3 and the porous foam soft pad 1. The tail end of the drainage tube 2 is connected with a drainage container 5 and a vacuum source 6 through a catheter 4. The tail end of the drainage tube 2 extends out from the upper side of the porous foam soft pad 1.

A flushing pipe 7 is arranged in the drainage tube 2, one end of the flushing pipe 7 extends to the inside of the drainage tube 2, the other end of the flushing pipe 7 is led out from the side wall at the tail end of the drainage tube 2, and a sealing cover 7a is arranged at the tail end of the flushing pipe 7.

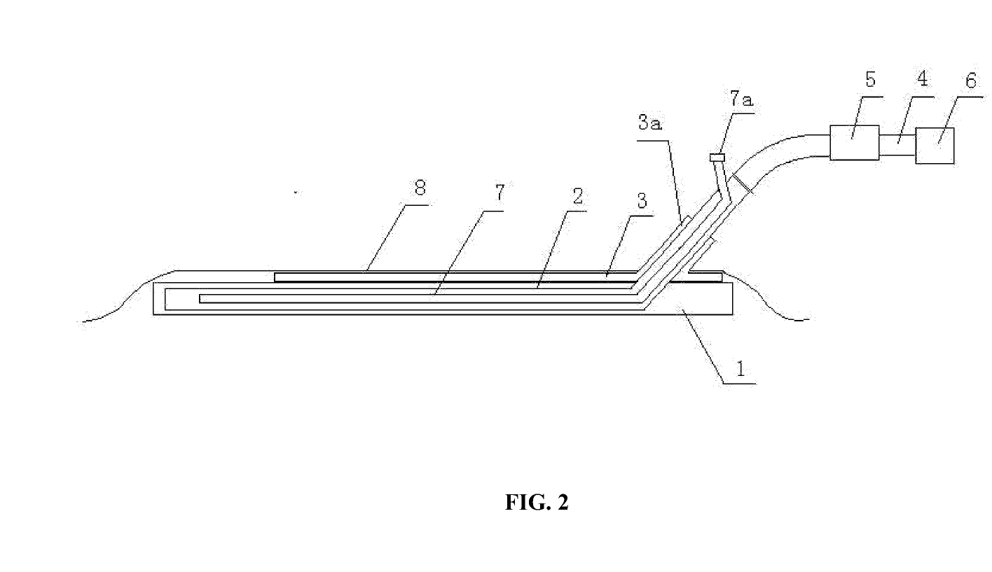

FIG. 2 is an overall structure diagram of a second basic vacuum sealing drainage device. The second basic vacuum sealing drainage device has the same basic structure as the first basic vacuum sealing drainage device, however, the sealing opening 3a used for allowing the drainage tube 2 to extend out and formed on the elastic sealing rubber film 3 is a pipe orifice, and the diameter of the opening part of the pipe orifice is 0.3-2 mm less than the outer diameter of a corresponding extending pipe.

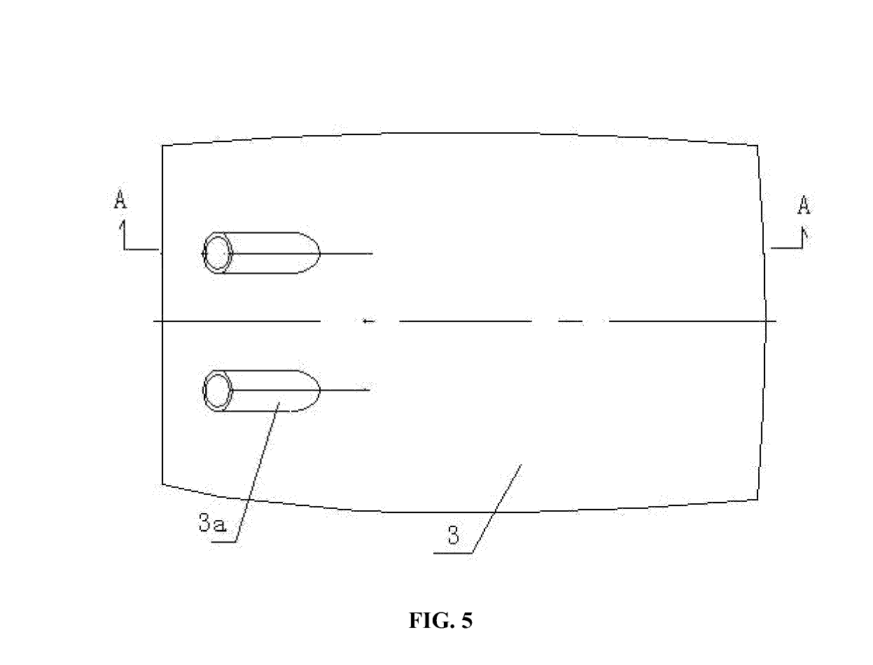

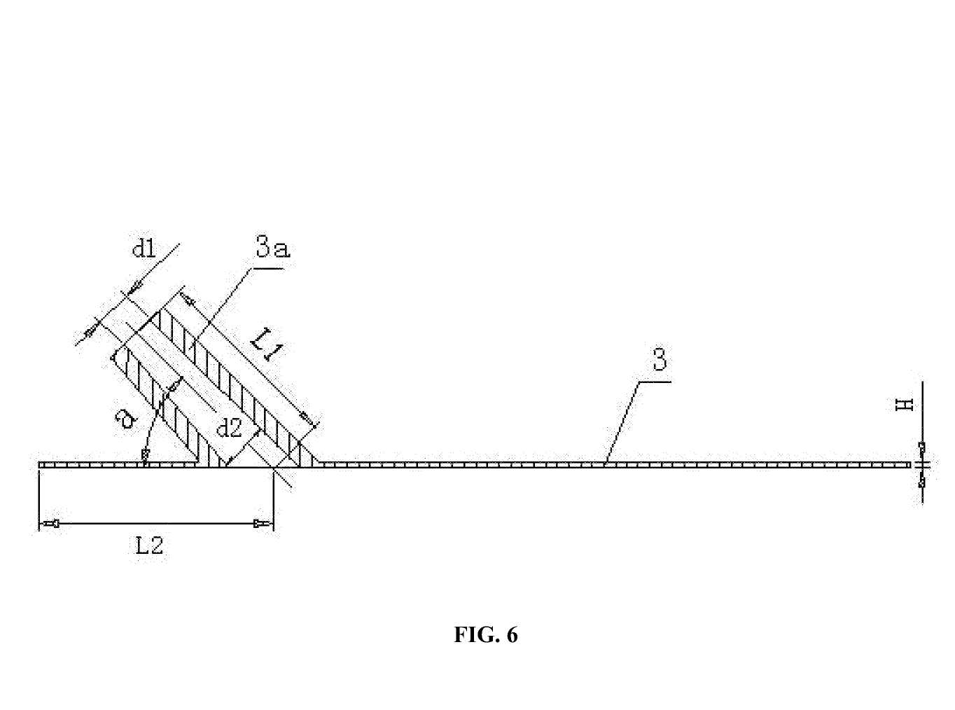

As shown in FIG. 5 and FIG. 6, the sealing opening 3a used for allowing tubes to extend out and formed on the elastic sealing rubber film 3 is a pipe orifice, the pipe orifice takes the shape of an inverted horn, the diameter d1 of the opening part of the pipe orifice is 0.3-2 mm less than the outer diameter of the drainage tube 2, and the diameter d2 of the bottom of the pipe orifice is 2-6 mm more than the outer diameter of the drainage tube 2. The included angle α between the sealing opening 3a and a main body of the elastic sealing rubber film 3 is between 15 and 90°, and preferably 30-45°.

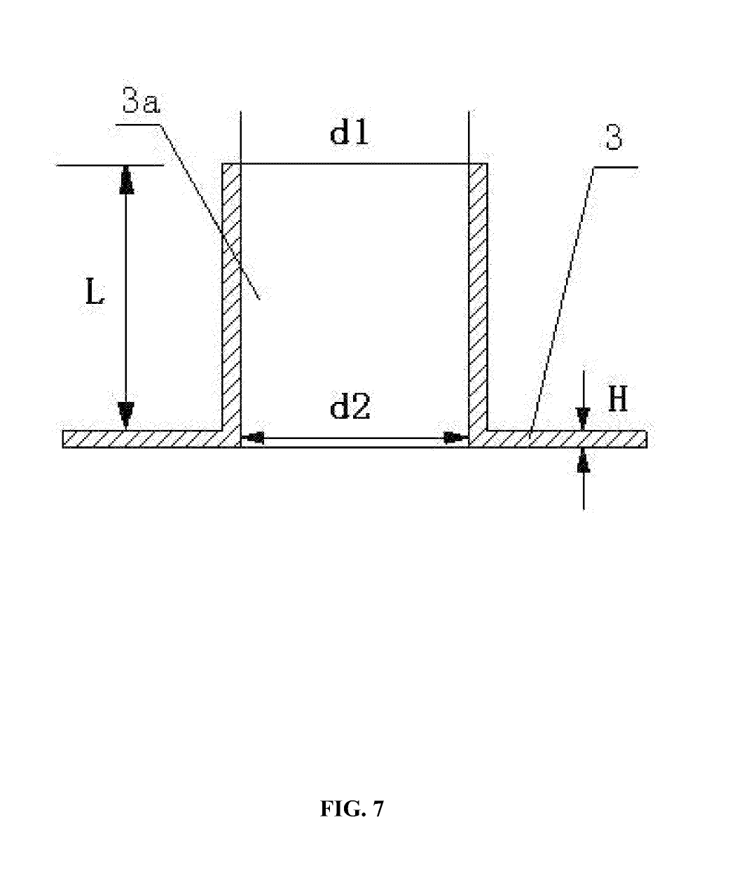

FIG. 7 is a diagram of a vertical pipe orifice shaped sealing opening on the elastic sealing rubber film. The included angle α between the sealing opening 3a and the main body of the elastic sealing rubber film 3 is 90°. The embodiment is suitable for vertical tubes.

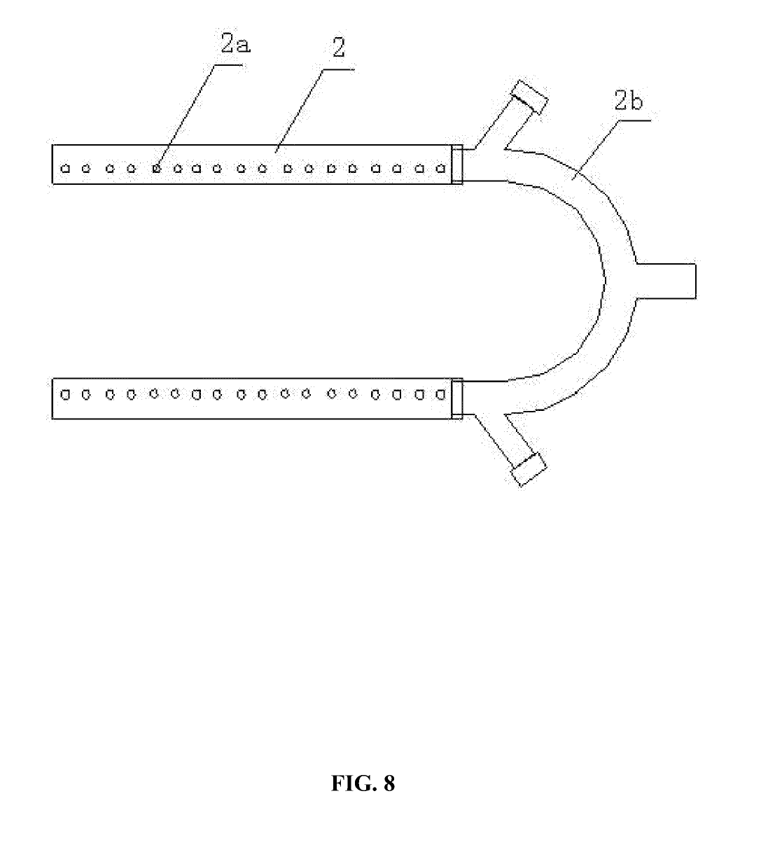

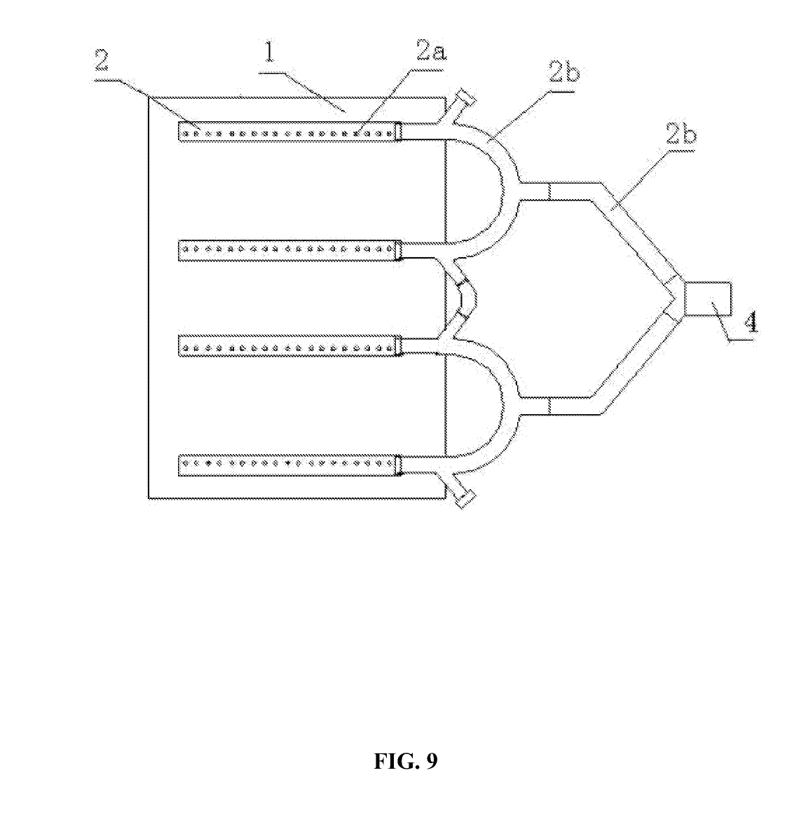

FIG. 8 is diagram of the drainage tube and a two-way connector mechanism for two drainage tubes. FIG. 9 is a diagram of four drainage tubes and a four-way connector mechanism which are arranged in the porous foam soft pad respectively.

A plurality of small holes 2a is formed on the side wall of the drainage tube 2. A plurality of drainage tubes are connected with a multi-way connector 2b, and the multi-way connector 2b is connected with the catheter 4. The drainage tubes are suitable for massive trauma. The plurality of the drainage tubes 2 are connected through the multi-way connector and then converged to enter the drainage container 5, and the operation is simple and convenient. For the use inside the body, a tube is lead out, thus the wound is small.

The structure solves the problem of sealing, thus the stable vacuum sealing drainage can be kept.

When the medical vacuum sealing drainage device is used for wounds on the body surface, as shown in FIG. 1 and FIG. 2, after the porous foam soft pad 1 is placed on the wound, the elastic sealing rubber film 3 is arranged at the position where the drainage tubes 2 extends out, and the sealing opening 3a is tightened on the drainage tubes 2 for sealing due to the elasticity of the elastic sealing rubber film, thus the elastic sealing rubber film has the sealing effect. The sealing film 8 covers the porous foam soft pad 1 and the elastic sealing rubber film 3 for sealing, so that the porous foam soft pad 1 is sealed with the body surface. Besides achieving the sealing effect, the elastic sealing rubber film covers the porous foam soft pad by increasing the area of the elastic sealing rubber film 3, so that the problem that if the sealing film 8 is directly stuck onto the porous foam soft pad 1, the porous foam soft pad may become hard due to the evaporation of water in the porous foam soft pad through the sealing film, and then the vacuum sealing drainage will fail is avoided.

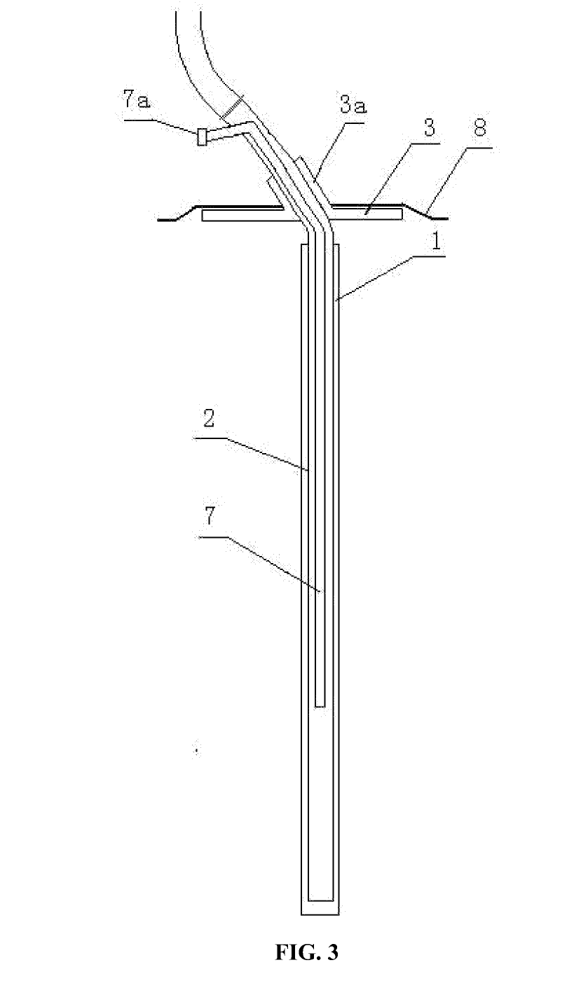

FIG. 3 is a diagram of the medical vacuum sealing drainage device used inside the body.

The example adopts the structure form that one end of the drainage tubes 2 is positioned in the porous foam soft pad 1, and the other end of the drainage tubes 2 is led out from the upper end of the porous foam soft pad 1. When the medical vacuum sealing drainage device is used in the body cavity, after the porous foam soft pad 1 is placed in the body cavity, the elastic sealing rubber film 3 is attached outside the body to provide an effective support (carrier) for the sealing film 8, and the sealing opening 3a is tightened on the drainage tube 2 for sealing due to the elasticity of the elastic sealing rubber film, thus the sealing effect is achieved. The sealing film 8 covers the elastic sealing rubber film 3, and the periphery of the elastic sealing rubber film 3 is sealed with the body surface.

When the small holes of the drainage tube 2 are blocked, a vacuum system is closed, the sealing cover 7a at the tail end of the flushing pipe 7 is opened, an irrigator is accessed, and normal saline is injected, so that stagnant blood clots are diluted and dissolved. After stopping washing, the vacuum system is opened, and the diluted stagnant blood clots are drawn out.

The current commonly-used washing method is as follows: (1) a rubber hose is additionally arranged at the wound, but the lead-out position of the rubber hose is also required to be sealed by a film sticking method, thus the possibility of air leakage and the sealing difficulty are increased; (2) when flushing fluid is retrogradely injected from the original drainage tube, the drained exudate may be reinjected to the wound to lead to retrograde infection; (3) when flushing fluid is injected through holes perforated on the porous foam soft pad, the sealing film is only required to be restuck at the perforated hole on the porous foam soft pad, thus the operating difficulty is increased.

In the medical vacuum sealing drainage device, the blocked pipe is conveniently flushed, the phenomenon of retrograde infection is avoided, and the flushing pipe does not occupy the space of the drainage tube. The sealing cover 7a is arranged at the tail end of the flushing pipe 7, and when the flushing pipe 7 is not used, the sealing cover is closed, so as to ensure the normal work of the drainage tube 2. The flushing pipe 7 also can be used as a sampling opening, and the drainage fluid is tested as the reference for medication for doctors.

A plurality of drainage tubes 2 are connected through a multi-way connector, when one drainage tube is blocked, observation can be carried out conveniently from a catheter connector, so that the blocked drainage tube is conveniently flushed in time through a flushing pipe, other drainage tubes can be continued to conduct drainage, and the phenomenon that the vacuum sealing drainage device cannot work normally after one drainage tube is blocked is avoided.

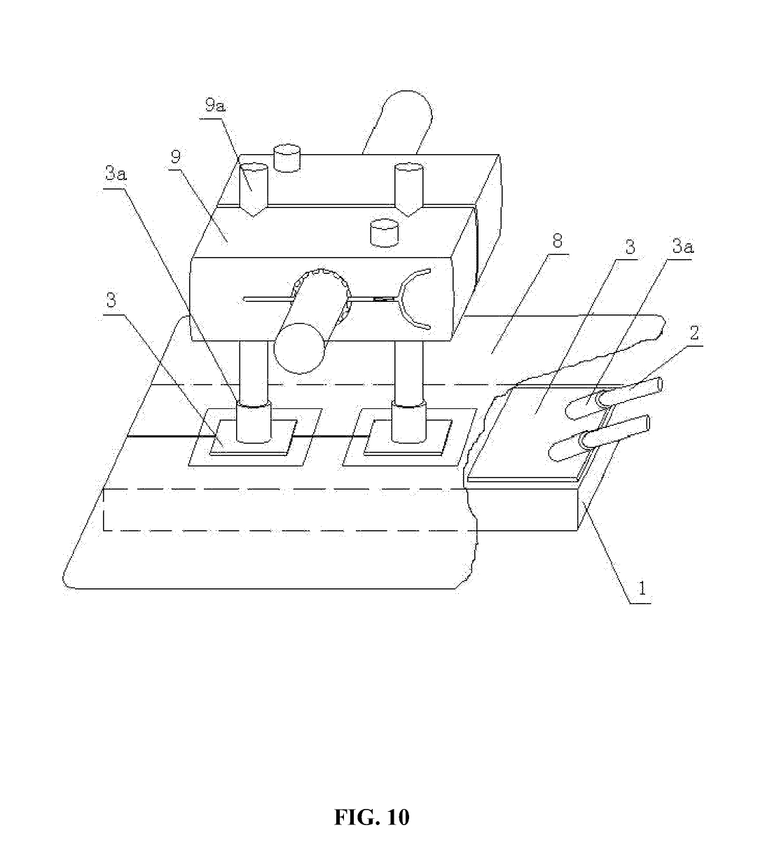

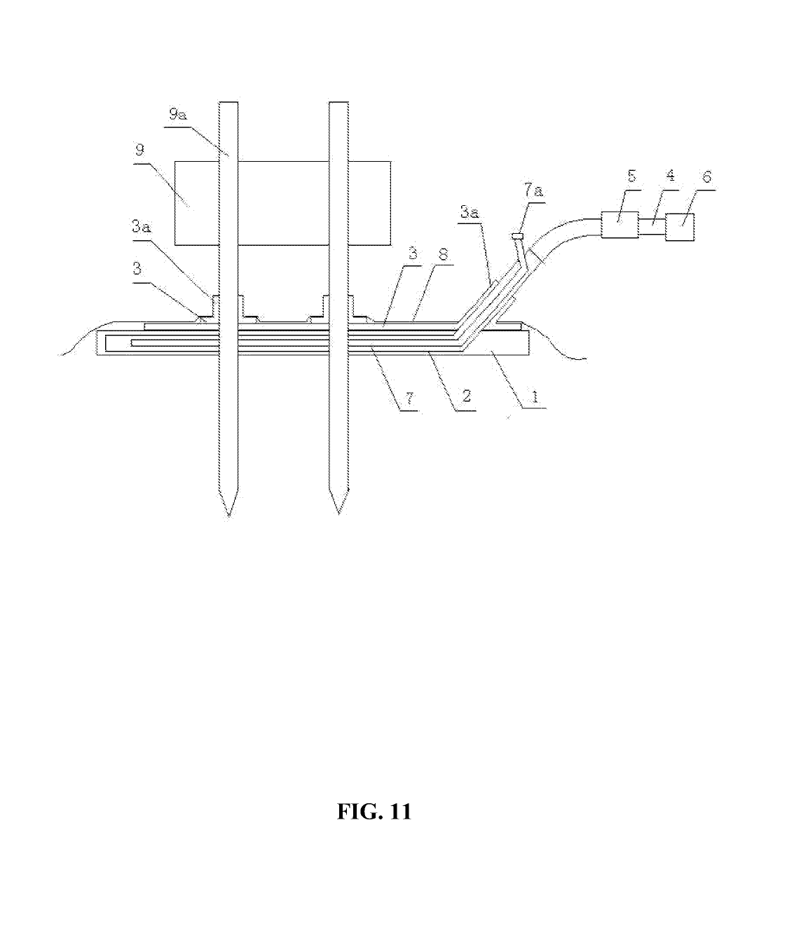

FIG. 10 and FIG. 11 are overall structure diagrams of orthopedic vacuum sealing drainage devices.

The orthopaedic vacuum sealing drainage device has the basically same structure as the basic vacuum sealing drainage devices, and comprises a porous foam soft pad 1, and a drainage tube 2 arranged in the porous foam soft pad 1 and provided with a tail end extending outside the porous foam soft pad 1, a first elastic sealing rubber film 3 is arranged on the outer side of the porous foam soft pad 1, a sealing opening 3a for allowing the drainage tube 2 to extend out is formed on the first elastic sealing rubber film 3, the diameter D of the opening part of the sealing opening 3a is smaller than the outer diameter of the corresponding drainage tube 2, a second elastic sealing rubber film 3 and a third elastic sealing rubber film 3 are arranged at puncture needles 9a connected with an orthopaedic external fixation device 9, vertical pipe orifice shaped sealing openings 3a are formed on the second elastic sealing rubber film 3 and the third elastic sealing rubber film 3, and sleeved on the two puncture needles 9a respectively, and the second and the third elastic sealing rubber films 3 are placed on the first elastic sealing rubber film 3 or directly placed on the porous foam soft pad 1 for sealing in a covering manner. A flushing pipe 7 is arranged in the drainage tube 2, one end of the flushing pipe 7 extends to the inside of the drainage tube 2, the other end of the flushing pipe 7 is led out from the side wall at the tail end of the drainage tube 2, and a sealing cover 7a is arranged at the tail end of the sealing cover 7a. The second and the third elastic sealing rubber films 3 are used to adapt the distance change of the two puncture needles 9a. When the sealing opening 3a used for allowing the drainage tube 2 to extend out and formed on the first elastic sealing rubber films 3 is a pipe orifice, the included angle α between the pipe orifice and a main body of the elastic sealing rubber film 3 is between 15 and 90°, and preferably 30-45°.

The pipe orifice shaped sealing opening 3a preferably takes the shape of an inverted horn. The diameter dl of the opening part of the pipe orifice is 1-2 mm less than the outer diameter of the drainage tube 2, and the diameter d2 of the bottom of the pipe orifice is 2-6 mm more than the outer diameter of the drainage tube 2.

The sealing openings 3a on the second and the third elastic sealing rubber films 3 are vertical pipe orifice shaped sealing openings 3a shown in FIG. 7, belong to the embodiment that the included angle α between the pipe orifice and the main body of the elastic sealing rubber films 3 is 90°, and used for sealing other tubes. The pipe orifice shaped sealing opening 3a takes the shape of an inverted horn, the diameter d1 of the opening part of the pipe orifice is 1-2 mm less than the outer diameter of the puncture needles 9a, and the diameter d2 of the bottom of the pipe orifice is 2-6 mm more than the outer diameter of the corresponding puncture needles 9a. The orthopaedic vacuum sealing drainage device changes the operating instructions and rules of the traditional orthopaedic external fixation technology and combines the sealing and fixing operation, and the orthopaedic external fixation device is hermetically sealed, so that the problem of failure or reduced effect in vacuum sealing drainage treatment due to air leakage is eliminated.

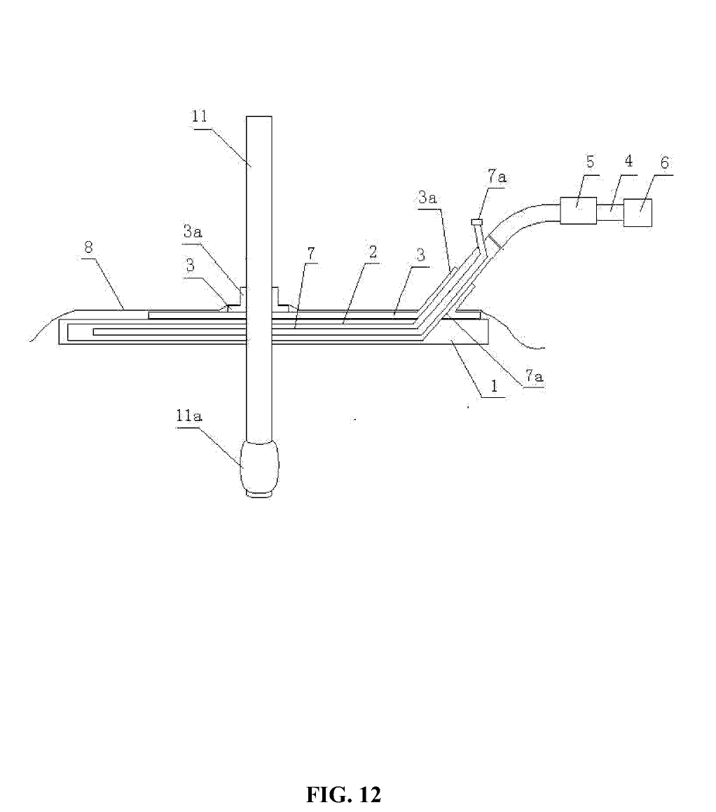

FIG. 12 is an overall structure diagram of a vacuum sealing drainage device used at the perineum.

The vacuum sealing drainage device used at the perineum has the basically same structure as the basic vacuum sealing drainage devices, and comprises a porous foam soft pad 1, and a drainage tube 2 arranged in the porous foam soft pad 1 and provided with a tail end extending outside the porous foam soft pad 1, a first elastic sealing rubber film 3 is arranged on the outer side of the porous foam soft pad 1, a sealing opening 3a for allowing the drainage tube 2 to extend out is formed on the first elastic sealing rubber film 3, and the diameter of the opening part of the sealing opening 3a is smaller than the outer diameter of the corresponding drainage tube 2. The vacuum sealing drainage device used at the perineum further comprises an anus inserting catheter 11, a second elastic sealing rubber film 3 is arranged at the position where the anus inserting catheter 11 penetrates a vacuum area of the vacuum sealing drainage device, and provided with a vertical pipe orifice shaped sealing opening 3a, the vertical pipe orifice shaped sealing opening 3a is sleeved on the anus inserting catheter 11, and the second elastic sealing rubber film 3 is placed on the first elastic sealing rubber film 3 or directly placed on the porous foam soft pad 1 for sealing in a covering manner. A flushing pipe 7 is arranged in the drainage tube 2, one end of the flushing pipe 7 extends to the inside of the drainage tube 2, the other end of the flushing pipe 7 is led out from the side wall at the tail end of the drainage tube 2, and a sealing cover 7a is arranged at the tail end of the flushing pipe 7.

The pipe orifice shaped sealing opening 3a of the second elastic sealing rubber film 3 preferably takes the shape of an inverted horn, the diameter d1 of the opening part of the pipe orifice is less than 1-2 mm less than the outer diameter of the anus inserting catheter 11, and the diameter d2 of the bottom of the pipe orifice is 2-6 mm more than the corresponding anus inserting catheter 11.

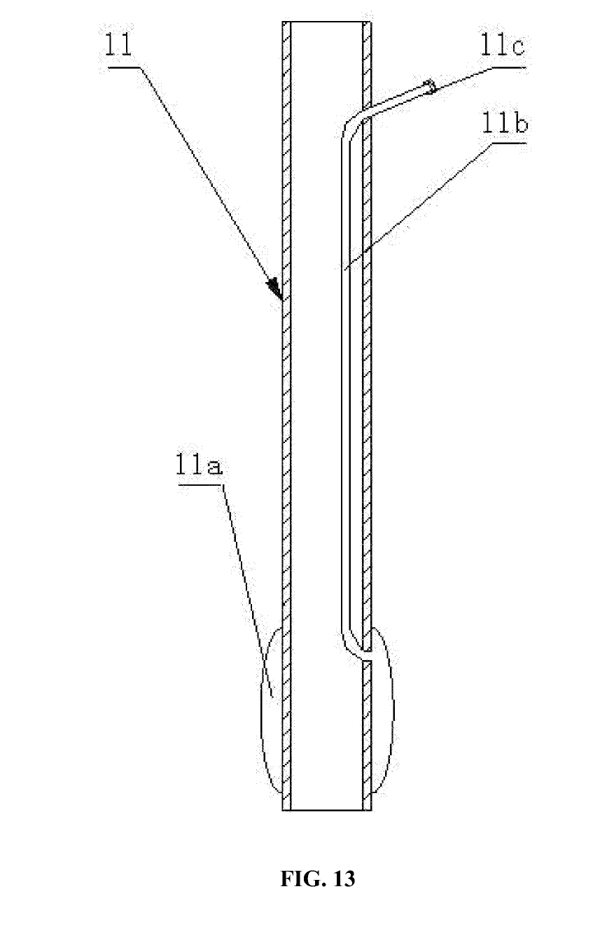

As shown in FIG. 13, an annular rubber air-filled or liquid-filled bag 11a is arranged on the outer side at the front end of the anus inserting catheter 11, an air or liquid guide pipe 11b parallel to the anus inserting catheter 11 is also arranged, the front end of the air or liquid guide pipe 11b is communicated with the rubber air-filled or liquid-filled bag 11a, the rear end of the air or liquid guide pipe 11b extends outside the rear end of the anus inserting catheter 11, and a sealing cover 11e is arranged at the rear end of the air or liquid guide pipe 11b. The liquid guide pipe 11b also can be arranged on the inner wall of the anus inserting catheter 11. In the VSD (vacuum sealing drainage) treatment, the anus inserting catheter is additionally arranged, so that the normal physiological excretion of patients is not affected, the excrement is effectively prevented from overflowing to pollute the wound, the incidence rate of infection is effectively lowered, and the wound healing time of patients is reduced.

In conclusion, skillfully solving the difficult problem (air leakage) unsolved for a long time through sealing the puncture tube part of the vacuum sealing drainage device under the elasticity of an elastic material is the core of the vacuum sealing drainage device. In a set of the vacuum sealing drainage devices, one or more can be used as required, and the vacuum sealing drainage devices with different sizes and shapes can be used alternately, thus the vacuum sealing drainage device is extremely flexible to use. Therefore, under the premise of without changing the principle of the vacuum sealing drainage device, any changes or deformations fall within the protection scope of the invention.

The invention claimed is:

1. A medical vacuum sealing drainage device, comprising:
a) a porous foam soft pad (1);
b) a drainage tube (2) comprising a tail end; and
c) an elastic sealing rubber film (3); wherein
the drainage tube (2) is arranged in the porous foam soft pad (1) and the tail end thereof extends outside the porous foam soft pad (1);
the elastic sealing rubber film (3) is arranged on an outer side of the porous foam soft pad (1);
a sealing opening (3a) for allowing the drainage tube (2) to extend out is formed on the elastic sealing rubber film (3); and
a diameter of an opening part of the sealing opening (3a) is less than an outer diameter of a corresponding extending pipe.

2. The device of claim 1, wherein the sealing opening (3a) is a circle hole, the diameter of which is 0.3-4 mm less than the outer diameter of the corresponding extending pipe.

3. The device of claim 1, wherein the sealing opening (3a) is a pipe orifice, and the diameter of the opening part of the pipe orifice is 0.3-2 mm less than the outer diameter of the corresponding extending pipe.

4. The device of claim 3, wherein the pipe orifice takes a shape of an inverted horn, the diameter of the opening part of the pipe orifice is 1-2 mm less than the outer diameter of the corresponding extending pipe, and the diameter of the bottom of the pipe orifice is 2-6 mm more than the outer diameter of the corresponding extending pipe.

5. The device of claim 4, wherein an included angle between the sealing opening (3a) and a main body of the elastic sealing rubber film (3) is between 15 and 90°.

6. The device of claim 1, wherein a plurality of small holes (2a) are formed on a side wall of the drainage tube (2).

7. The device of claim 3, wherein a plurality of small holes (2a) are formed on a side wall of the drainage tube (2).

8. The device of claim 1, wherein a single or a plurality of drainage tubes (2) are employed; the plurality of drainage tubes (2) are arranged evenly with a distance of 25-35 mm between each other, and connected through a multi-way connector.

9. The device of claim 3, wherein a single or a plurality of drainage tubes (2) are employed; the plurality of drainage tubes (2) are arranged evenly with a distance of 25-35 mm between each other, and connected through a multi-way connector.

10. The device of claim 1, wherein a flushing pipe (7) is arranged in the drainage tube (2), one end of the flushing pipe (7) extends to the inside of the drainage tube (2), the other end of the flushing pipe (7) is led out from a side wall at the tail end of the drainage tube (2), and a sealing cover (7a) is arranged at the tail end of the flushing pipe (7).

11. The device of claim 3, wherein a flushing pipe (7) is arranged in the drainage tube (2), one end of the flushing pipe (7) extends to the inside of the drainage tube (2), the other end of the flushing pipe (7) is led out from a side wall at the tail end of the drainage tube (2), and a sealing cover (7a) is arranged at the tail end of the flushing pipe (7).

12. An orthopedic vacuum sealing drainage device, comprising:
a) a porous foam soft pad (1);
b) a drainage tube (2) comprising a tail end;
c) a first elastic sealing rubber film (3);
d) a second elastic sealing rubber film (3); and
e) a third elastic sealing rubber film (3); wherein
the drainage tube (2) is arranged in the porous foam soft pad (1) and the tail end thereof extends outside the porous foam soft pad (1);
the first elastic sealing rubber film (3) is arranged on an outer side of the porous foam soft pad (1);
a sealing opening (3a) for allowing the drainage tube (2) to extend out is formed on the first elastic sealing rubber film (3);
a diameter of an opening part of the sealing opening (3a) is smaller than an outer diameter of a corresponding drainage tube (2);
the second elastic sealing rubber film (3) and the third elastic sealing rubber film (3) are arranged at puncture needles (9a) connected with an orthopaedic external fixation device (9), vertical pipe orifice shaped sealing openings (3a) are formed on the second elastic sealing rubber film (3) and the third elastic sealing rubber film (3), and sleeved on the two puncture needles (9a) respectively; and
the second and the third elastic sealing rubber films (3) are placed on the first elastic sealing rubber film (3) or directly placed on the porous foam soft pad (1) for sealing in a covering manner.

13. The device of claim 12, wherein the sealing opening (3a) is a pipe orifice, and an included angle between the pipe orifice and a main body of the elastic sealing rubber film (3) is between 15 and 90°.

14. The device of claim 12, wherein the vertical pipe orifice shaped sealing opening on the second and the third elastic sealing rubber films (3) takes a shape of an inverted horn, the diameter of the opening part of the pipe orifice is 0.3-2 mm less than the outer diameter of the puncture needles (9a), and the diameter of the bottom of the pipe orifice is 2-6 mm more than the outer diameter of the corresponding puncture needles (9a).

15. The device of claim 12, wherein a flushing pipe (7) is arranged in the drainage tube (2), one end of the flushing pipe (7) extends to the inside of the drainage tube (2), the other end of the flushing pipe (7) is led out from the side wall at the tail end of the drainage tube (2), and a sealing cover (7a) is arranged at the tail end of the sealing cover (7a).

16. The device of claim 13, wherein a flushing pipe (7) is arranged in the drainage tube (2), one end of the flushing pipe (7) extends to the inside of the drainage tube (2), the other end of the flushing pipe (7) is led out from the side wall at the tail end of the drainage tube (2), and a sealing cover (7a) is arranged at the tail end of the sealing cover (7a).

17. A vacuum sealing drainage device used at perineum, comprising:
a) a porous foam soft pad (1);
b) a drainage tube (2) comprising a tail end;
c) a first elastic sealing rubber film (3);
d) an anus inserting catheter (11); and
e) a second elastic sealing rubber film (3); wherein
the drainage tube (2) is arranged in the porous foam soft pad (1) and the tail end thereof extends outside the porous foam soft pad (1);
the first elastic sealing rubber film (3) is arranged on an outer side of the porous foam soft pad (1);
a sealing opening (3a) for allowing the drainage tube (2) to extend out is formed on the first elastic sealing rubber film (3);
a diameter of an opening part of the sealing opening (3a) is smaller than an outer diameter of a corresponding drainage tube (2);
the second elastic sealing rubber film (3) is arranged at the position where the anus inserting catheter (11) penetrates a vacuum area of the vacuum sealing drainage device, and provided with a vertical pipe orifice shaped sealing opening, the vertical pipe orifice shaped sealing opening is sleeved on the anus inserting catheter (11); and
the second elastic sealing rubber film (3) is placed on the first elastic sealing rubber film (3) or directly placed on the porous foam soft pad (1) for sealing in a covering manner.

18. The device of claim 17, wherein an annular rubber air-filled or liquid-filled bag (11a) is arranged on the outer side at a front end of the anus inserting catheter (11);
an air or liquid guide pipe (11b) parallel to the anus inserting catheter (11) is also arranged; and
the front end of the air or liquid guide pipe (11b) is communicated with the rubber air-filled or liquid-filled bag (11a), the rear end of the air or liquid guide pipe (11b) extends outside the rear end of the anus inserting catheter (11), and a sealing cover (11c) is arranged at the rear end of the air or liquid guide pipe (11b).

19. The device of claim 17, wherein a flushing pipe (7) is arranged in the drainage tube (2), one end of the flushing pipe (7) extends to the inside of the drainage tube (2), the other end of the flushing pipe (7) is led out from the side wall at the tail end of the drainage tube (2), and a sealing cover (7a) is arranged at the tail end of the flushing pipe (7).

20. The device of claim 18, wherein a flushing pipe (7) is arranged in the drainage tube (2), one end of the flushing pipe (7) extends to the inside of the drainage tube (2), the other end of the flushing pipe (7) is led out from the side wall at the tail end of the drainage tube (2), and a sealing cover (7a) is arranged at the tail end of the flushing pipe (7).
